(19) United States
(12) Patent Application Publication
(10) Pub. No.: US 2024/0423778 A1
(43) Pub. Date: Dec. 26, 2024

(54) PRESSURE REGULATED VEIN GRAFT PREPARATION PUMP SYSTEM AND METHOD OF USE

(71) Applicant: Quest Medical, Inc., Allen, TX (US)

(72) Inventors: Steve Parnis, Missouri City, TX (US); Andrew Nelson, Dallas, TX (US); Jeffrey Albertsen, McKinney, TX (US)

(21) Appl. No.: 18/749,907

(22) Filed: Jun. 21, 2024

Related U.S. Application Data
(60) Provisional application No. 63/522,579, filed on Jun. 22, 2023.

(51) Int. Cl. A61F 2/06

(52) U.S. Cl. CPC A61F 2/062

(57) ABSTRACT

A pressure-regulated vein graft preparation pump for preparing a vein graft. The pressure-regulated vein graft preparation pump includes: a source of pressurized fluid which is configured to supply the pressurized fluid through a flow path extending through a fluid conduit, a cannula configured to be coupled to an end of the fluid conduit, and a pressure control device coupled to the cannula. The pressure control device is located downstream from the source of the pressurized fluid and configured to limit a pressure exerted on the fluid conduit by the pressurized fluid.

### CROSS-REFERENCE TO RELATED APPLICATIONS

This application claims the benefit of U.S. Application Ser. No. 63/522,579 filed on Jun. 22, 2023, the entirety of which is incorporated by reference.

### BACKGROUND

### Technical Field

The present disclosure is generally related to the field of vein grafting and more particularly to a novel pressure regulated vein graft preparation pump for preparing veins for grafting, as well as methods of use and methods of manufacture thereof.

### Description of Related Art

Coronary artery disease (CAD) is caused by the buildup of plaque in the arteries of the heart, which results in stenotic lesions (i.e., narrow or constricted areas) in cardiac arteries that can impede normal blood flow. There are two methods by which stenotic lesions of the cardiac arteries are revascularized: percutaneous coronary intervention (PCI) and coronary artery bypass surgery, also called coronary artery bypass grafting (CABG). PCI is a non-surgical procedure used to treat narrowing of the coronary arteries using a combination of coronary angioplasty and stenting. Conversely, a CABG procedure is a surgical procedure using arteries or veins harvested from other parts of the body to bypass narrowing areas of the coronary arteries. After performing such a bypass, CABG restores adequate blood supply to the heart, thereby slowing the progression of CAD and increasing life expectancy for the patient.

A common CABG procedure involves a saphenous vein graft (SVG) for the bypassing of narrowed areas of the arteries. The great saphenous vein (or long saphenous vein) is a large, subcutaneous, superficial vein of the leg. It is the longest vein in the body, running along the length of the lower limb for returning blood from the foot, leg, and thigh to the deep femoral vein. Unfortunately, long-term patency (i.e., remaining sufficiently open) of SVGs is a common problem and vein graft failure (VGF) reportedly as high as 45% at 18 months after surgery. It is believed that the high long-term failure rate is due to injury of the SVG during preparation prior to grafting, which may promote VGF.

Several components of the vein harvesting and engraftment process result in damage to the tunica intima (i.e., the innermost layer) of vein grafts. For example, veins are damaged during harvesting and engraftment due to ischemia (restriction in blood flow/supply) and reperfusion (return of blood flow/supply following a period of lack of oxygen). During and after harvesting, veins go through a period of ischemia. After engraftment, veins go through reperfusion. Both ischemia and reperfusion damage endothelial cells (cells that line the interior surface of the vein) and smooth muscle cells (the cells present in the layer surrounding the tunica intima).

Veins are also damaged during engraftment due to exposure to an environment to which they are not naturally adapted. Veins are naturally adapted to an environment of low pressure and low blood flow. During vein grafting, veins are exposed to high pressure and flow as they are transferred and integrated into the arterial circulation. This exposure to high pressure and flow causes increases shear stress and wall tension, which further damages the endothelial layer and smooth muscle cells. Over time, such continued damage results in luminal loss that makes the graft more susceptible to atherosclerosis. Progressive atherosclerosis is the primary cause of late vein graft failure.

Some structural changes of the tunica intima are necessary during preparation of the graft to prevent thickening (i.e., intimal hyperplasia) and vein wall remodeling after engraftment. Procedures to accomplish these structural changes typically involve distension (i.e., stretching) of the veins using fluid pressure. When elevated or unregulated pressure is used, these preparation procedures can also contribute to vein graft damage and VGF.

Conventional preparation of saphenous veins for an SVG typically involves the manual distension of the veins using fluid pressure. For example, U.S. Pat. No. 3,958,557 provides a device and method for preparing a blood vessel, such as the saphenous vein, for use as a coronary bypass graft. FIG. 1 illustrates an isometric view of a cannula 10 constructed in accordance with the principles disclosed in the U.S. Pat. No. 3,958,557. This conventional cannula 10 is comprised of a medical grade polyethylene having the flexibility and softness required to prevent trauma to the intima of the vein in which it is used. The cannula 10 has a hub 14 at the proximal end thereof which is cylindrical and terminates in a planar surface 16. The hub 14 is connected to a body 12 that is diametrically enlarged to form a shoulder 18 in a plane parallel to the surface 16. The hub 14 is sized to accommodate coupling with surgical tubing (not shown), which is press-fit on the hub 14 and abuts shoulder 18. The body 12 is frustoconical in configuration, the forwardly tapering ramp surface 20 merging with stylet 22, which forms the distal end of the cannula 10. The stylet 22 has a very gradual taper from the body 12 to the distal end 24. The external dimension of the stylet 22 is selected to be essentially the normal internal diameter of a suitable vein segment for use in coronary bypass. The stylet 22 includes a peripheral flange 26 spaced slightly behind the distal end 24. The peripheral flange 26 has a forwardly tapering ramp surface 28 that facilitates insertion of the stylet 22 into a suitable vein segment 30. The flange 26 terminates in a shoulder 32 which permits pressure-tight ligation, as described below.

FIG. 2 illustrates a side, partial cross-section view of the conventional cannula 10 taught in U.S. Pat. No. 3,958,557. The stylet 22 has an annular bore 34 that opens at the distal end 24 and is also in open communication with hollow 38 in the hub 14. The hollow 38 is coaxial with the bore 34 and is provided with a Luer taper so as to form a female coupling. Thus, the cannula 10 is press-coupled to an irrigation instrument such as a syringe 40. The selected portion of the vein 30 is then severed and the stylet 22 inserted within the lumen of the resected vein 30 until the vein 30 is pressed tightly upon the ramp surface 20. Thereafter, temporary ligation is effected by tying suture 42 tightly around the vein 30 near the flange 26. Ligation will have the effect of creating a fluid seal between the vein segment 30 and the cannula 10.

Thereafter, a syringe 40 is used for irrigating purposes to determine whether occlusions or clots remain in the vein segment 30 and, if so, to accommodate flushing of the vein 30. A distant portion of the vein segment 30 can be clamped and manual hydrostatic pressure communicated to the vein through the cannula 10 with the syringe 40. This applied pressure is used to detect leaks in the vein segment 30, particularly at the severed tributary sites. This pressure also serves to determine the distensibility index of the vein 30. If the vein 30 distends too easily under pressure, it is not suitable for the coronary artery bypass. Assuming the vein 30 has been proven under this manual pressurization, the vein 30 is used for the coronary artery bypass.

However, data has shown that acute damage to the vein graft intima can result from extensive distension of such veins during repair of the severed tributaries of the vein graft, but that this does not necessarily occur at pressure equivalent to normal arterial pressure. However, manual pressure distension does not limit or otherwise regulate the pressure of fluid applied and does not circulate the fluid during preparation. Consequently, intimal damage, although unknown to the clinician at the time, is often caused to the grafted vein when prepared using such manual procedures due to high pressurization as a result of unregulated manual pressurization techniques found in U.S. Pat. No. 3,958,557 as well as other conventional approaches. Accordingly, what is needed in the art is a device and related method for limiting the pressure of the vein graft during preparation for a vein graft to be used in a coronary artery bypass procedure that does not suffer from the deficiencies found with conventional vein preparation approaches. The disclosed principles provide such a unique device, methods of use, and methods of manufacture thereof.

### BRIEF SUMMARY

This summary provides a discussion of aspects of certain embodiments of the invention. It is not intended to limit the claimed invention or any of the terms in the claims. The summary provides some aspects but there are aspects and embodiments of the invention that are not discussed here.

In one aspect, a pressure-regulated vein graft preparation pump is provided. The pressure-regulated vein graft preparation pump includes: a source of pressurized fluid which is configured to supply the pressurized fluid through a flow path extending through a fluid conduit, a cannula configured to be coupled to an end of the fluid conduit, and a pressure control device coupled to the cannula. The pressure control device is located downstream from the source of the pressurized fluid and configured to limit a pressure exerted on the fluid conduit by the pressurized fluid.

In yet another aspect, a method of operating an elastomeric vein graft preparation pump is provided. The method can include coupling a source of pressurized fluid to a flow path extending through a fluid conduit, coupling a cannula to an end of the fluid conduit, coupling a pressure control device to the cannula, introducing the pressurized fluid into the fluid conduit via the flow path. The pressure control device is located downstream from the source of the pressurized fluid and configured to limit a pressure exerted on the fluid conduit by the pressurized fluid.

Other aspects, embodiments and features of the invention will become apparent from the following detailed description of the invention when considered in conjunction with the accompanying figures. In the figures, each identical, or substantially similar component that is illustrated in various figures is represented by a single numeral or notation. For purposes of clarity, not every component is labeled in every figure. Nor is every component of each embodiment of the invention shown where illustration is not necessary to allow those of ordinary skill in the art to understand the invention.

### BRIEF DESCRIPTION OF THE DRAWINGS

The novel features believed characteristic of the disclosure are set forth in the appended claims. The disclosure itself, however, as well as a preferred mode of use, further objectives, and advantages thereof, will be best understood by reference to the following detailed description of illustrative embodiments when read in conjunction with the accompanying drawing, in which:

FIG. 1 illustrates an isometric view of a cannula constructed in accordance with the prior art, and used in the irrigation of a vein harvested for a grafting procedure;

FIG. 2 illustrates a side, partial cross-section view of the conventional cannula illustrated in FIG. 1;

FIG. 3 illustrates a perspective view of an exemplary embodiment of a pressure regulated vein graft preparation pump designed and constructed in accordance with the disclosed principles;

FIGS. 4A and 4B illustrate perspective views of one embodiment of a control valve for use with the pressure regulated vein graft preparation pump according to the present disclosure;

FIGS. 5A and 5B illustrate perspective views of an exemplary embodiment of a pressure control device for use with the pressure regulated vein graft preparation pump according to the present disclosure;

FIG. 6 illustrates a perspective view of an exemplary embodiment of a pressure regulated vein graft preparation pump designed and constructed in accordance with the disclosed principles; and

FIGS. 7A and 7B illustrate perspective views of an exemplary embodiment of a pressure control device for use with the pressure regulated vein graft preparation pump according to the present disclosure.

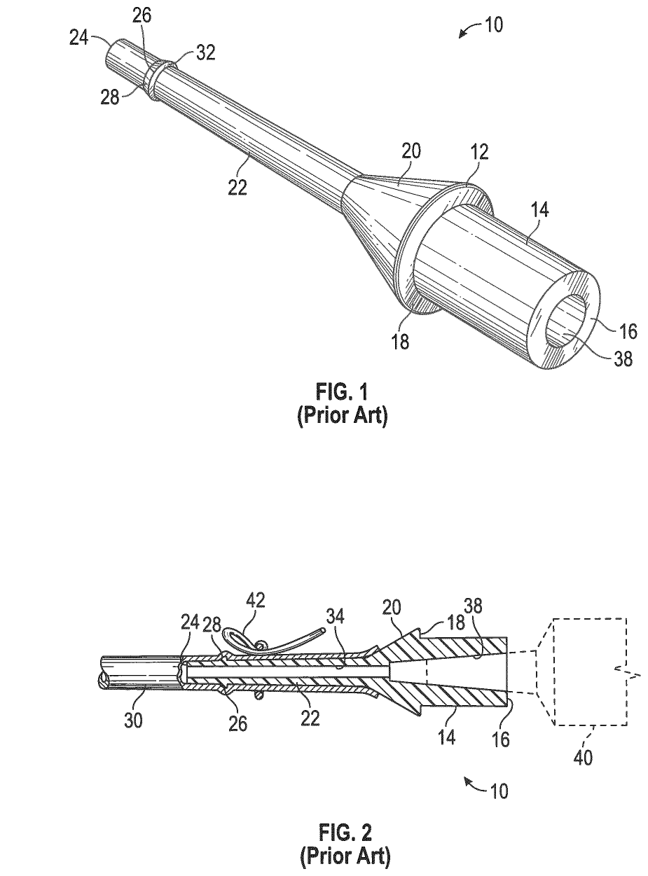

### DETAILED DESCRIPTION

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. Although multiple embodiments are shown and discussed in great detail, it will be apparent to those skilled in the relevant art that some features that are not relevant to the present invention may not be shown for the sake of clarity.

As used in this disclosure, the term “proximal” defines the syringe end of the described embodiments; that is, the axial direction toward the syringe. The term “distal” similarly defines the end of the described embodiments opposite the syringe; that is, the axial direction opposite that of the syringe. Furthermore, the terms “upstream” and “downstream” are relative terms based on the direction of fluid flow through the pressure-regulated vein graft preparation pump. For example, the upstream end of the fluid conduit is analogous to the proximal end of the fluid conduit and the downstream end of the fluid conduit is analogous to the distal end of the fluid conduit.

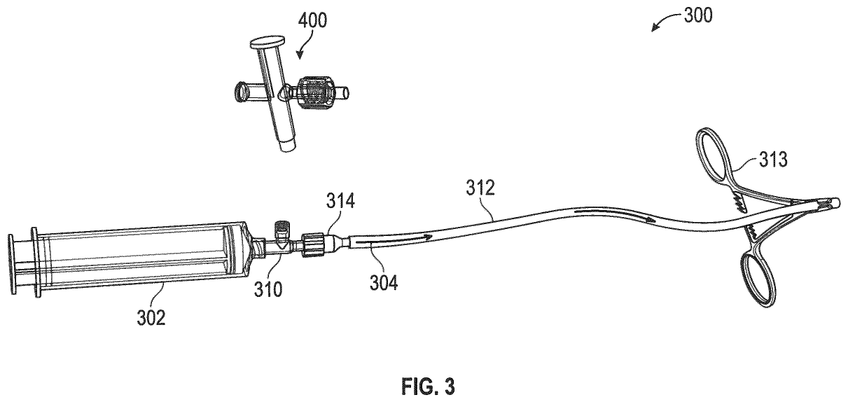

Referring to FIG. 3, an exemplary pressure regulated vein graft preparation pump 300 (hereinafter “pump 300”) is depicted, which is designed and constructed in accordance with the disclosed principles. As mentioned above, the pump 300 provides regulated pressurization of the fluid pushed through harvested veins. This pressurization at a predetermined amount safe for the harvested vein section eliminates the human error that too often causes over pressurization during distension of grafted veins due to manual pressurization.

The pump 300 includes a syringe 302 that is sized and configured to securely hold a predetermined amount of fluid and provide fluid under pressure for vein irrigation of vein 312 (alternatively referred to as a fluid conduit). The pressure of the supplied fluid is controlled by a pressure control device (alternatively referred to herein as valve) 310 disposed in the flow path extending from the syringe 302 and into the vein 312. The flow path, which is represented by arrow 304, may be referred to in the alternative as flow path 304. As can be seen, the flow path 304 is defined, at least in part, by the pressure control device 310, the cannula 314, and optionally the control valve 400 when included in pump 300. The control valve 400 can be inserted between the syringe 302 and the pressure control device 310. Alternatively, the control valve 400 can be inserted between the pressure control device 310 and the vein 312, e.g., between the pressure control device 310 and the cannula 314.

In the embodiment depicted in FIG. 3, the pressure control device 310 is a pressure regulator that controls the amount of pressure of the fluid being delivered downstream of the pressure control device 310, i.e., to the vein 312. In another embodiment, the pressure control device 310 is a pressure relief valve that permits the release of fluid in the event that the pump pressure exceeds the set point of the pressure control device 310, or the pressure control device 310 can be a combination of a pressure regulator and a pressure relief valve.

The pump 300 also includes a cannula 314 coupled to the pressure control device 310, which can be sutured or otherwise connected to the proximal end of the vein 312. The distal end of the vein 312 is shown sealed by a pair of hemostats 313 to maintain fluid within the vein 312.

In practice, a user can introduce the fluid from syringe 302 through pressure control device 310 and into vein 312, which is sealed with a clamp 313 (e.g., hemostat). As fluid is introduced into vein 312, the fluid pressure begins to increase. Once the pressure meets (or exceeds) the maximum pressure threshold (or threshold value) of the pressure control device 310, the pressure control device 310 terminates the flow of the fluid into the vein. The pressure control device 310 advantageously provides a safe mechanism for performing vein distension because the user can set the maximum pressure threshold (or threshold value) to a value that will not damage the vein. The maximum pressure threshold (or threshold value) can be between 2-4 PSI.

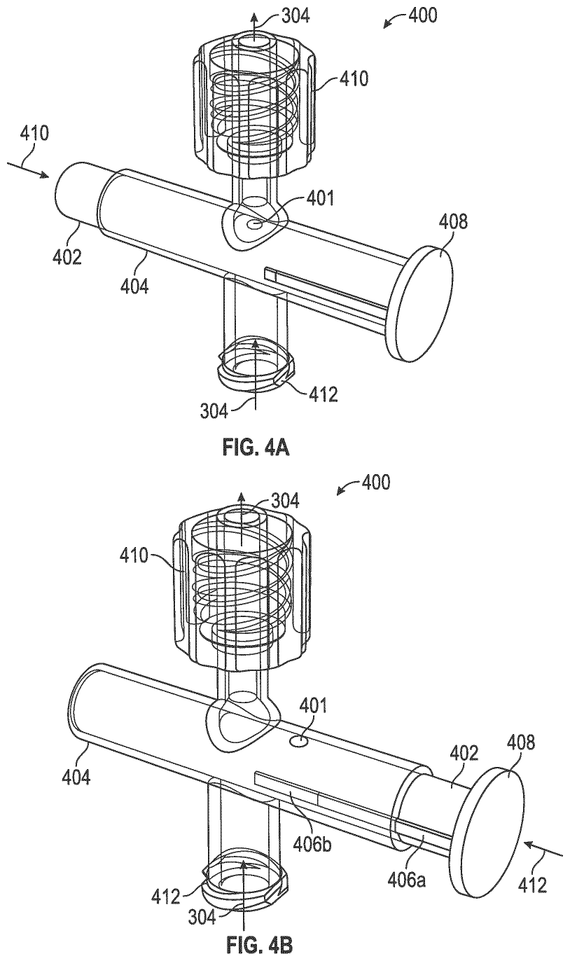

In some embodiments, the pump 300 includes an optional control valve 400 that controls the flow of fluid through the flow path 304. The control valve 400 is configured to allow the user of pump 300 to manually stop and start the flow of fluid through flow path 304. The control valve 400 can be used to prevent inadvertent discharge of fluid through the vein 312. A particular example of control valve 400 is shown in FIG. 4 that follows. Another exemplary control valve is control valve 108 that is shown and described in U.S. Application Ser. No. 63/522,577, the disclosure of which is incorporated herein in its entirety. Other examples of control valve 400 can include but are not limited to ball valves, gate valves, butterfly valves, and globe valves.

The illustrative embodiment depicted in FIG. 3 depicts the fluid source as a syringe 302, i.e., a vessel with a mechanically actuated plunger that can be used to control fluid flow through the fluid flow path 304. However, in another embodiment the syringe 302 can be replaced by a vessel configured to supply pressurized fluid, which obviates the need for the user to depress the plunger of the syringe 302. The control valve 400 can be used as a means to control the flow of fluid through the pump 300.

FIGS. 4A and 4B illustrate an exemplary embodiment of a control valve 400 for use with the pump 300. In the depicted embodiment, the control valve 400 includes a plug 402 with a plug aperture 401 that passes through the plug 402 from one side to the other. The plug 402 is slidably engaged within a valve plug body 404 so that the control valve 400 can be placed in the open position, as shown in FIG. 4A, or in the closed position, as shown in FIG. 4B. In an embodiment in which the plug 402 is cylindrical and the valve plug body 404 defines a cylindrical cavity sized to receive the plug 402, the control valve 400 can include an alignment interface 406 to prevent the valve plug 402 from rotating within the valve plug body 404. In this illustrated embodiment in FIGS. 4A and 4B, the alignment interface 406 includes an alignment rail 406a that is received within an alignment channel 406b.

The plug 402 can include a plug head 408, which can receive a pressing force in the direction of arrow 410 to cause the control valve 400 to transition from the closed configuration to the open configuration. In the depicted embodiment, the plug head 408 has a diameter that is larger than a diameter of the valve plug body 404 to limit travel of the plug 402 so that fully depressing the plug head 408 causes the aperture 401 to align with the flow path 304 that passes through the control valve 400.

Extending outwardly from the valve plug body 404 is a pair of connection interfaces 410 and 412. The connection interfaces 410 and 412 can be selected to engage with one of the syringe 302 or the pressure controller 310. Although connection interfaces 410 and 412 are depicted as screw-type interfaces, the examples shown in FIGS. 4A and 4B are exemplary and nonlimiting. Thus, the connection interfaces 410 and 412 can be any currently existing or later developed form of connector.

With reference now to FIG. 4A, the control valve 400 is in the open position when the control valve aperture 401 is aligned with the flow path 304. In the depicted embodiment, the control valve 400 is in the open position when the plug 402 is maximally depressed. The control valve 400 will remain in the open position until a force applied to the plug 402 in the direction of arrow 410 causes the control valve 400 to assume the closed position, wherein the control valve aperture 401 is not aligned flow path 304 as depicted in FIG. 4B. Alternatively, a grasping force applied to the plug head 408 and exerted in the general direction of arrow 410 can also cause the control valve 400 to assume the closed position.

With reference now to FIG. 4B, the control valve 400 is in the closed position when the control valve aperture 401 is not aligned with the flow path 304. The control valve 400 will remain in the closed position until a force applied to the plug 402 in the direction of arrow 414 causes the control valve 400 to assume the open position, wherein the control valve aperture 401 is aligned with the flow path 304 as depicted in FIG. 4A.

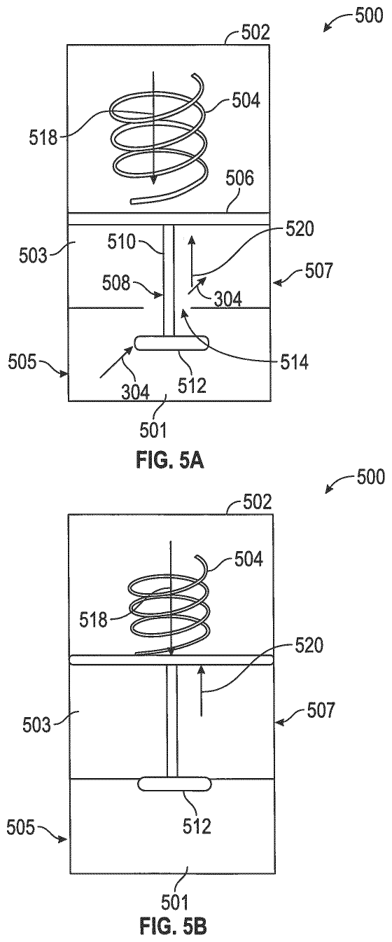

FIGS. 5A and 5B illustrate the principle of operation of a pressure control device for use with the pump 300. The illustrative pressure control device is a pressure regulator 500 that can be substituted in place of pressure control device 310 in FIG. 3 and used to control pressure downstream from the pressure regulator 500 by providing a variable internal volume that allows fluid flowing through the pressure regulator 500 to occupy a larger volume, which results in a reduction in downstream pressure. Pressure regulator 500 is meant to be exemplary and non-limiting.

In the depicted embodiment, the pressure regulator 500 includes a housing 502 that defines an upstream chamber 501 that is fluidically connected to the downstream chamber 503 by a pressure regulator passage 514. Fluid flow is controlled by a valve 508 in the housing 502. The valve 508 is formed from spring 504 connected to a spring base 506, which is in turn connected to a stem 510 with a plug 512 at the end. When the valve 508 is in the open position, fluid is able to flow into the upstream chamber 501 through an inlet 505, through the pressure regulator passage 514, into the downstream chamber 503 and out of the outlet 507. The flow path of fluid through the pressure regulator 500 is illustrated by arrows 304.

The spring 504 exerts a loading force against the spring base 506 in a direction represented generally by arrow 518 (hereinafter “spring force 518”) to separate the plug 512 from the pressure regulator passage 514 to maintain the valve 508 in the fully open position at baseline, i.e., in the absence of fluid pressure. The spring 504 is configured with a set spring constant (k) which determines the amount of force necessary to compress the spring 504 by a given amount. The spring constant is selected to limit fluid flow through the pressure regulator 500 to the desired or required maximum downstream fluid pressure.

When the valve 508 is in the closed position, as shown in FIG. 5B, the pressure regulator passage 514 is sealed by a plug 512 and fluid flow through the pressure regulator 500 is prevented. The valve 508 assumes the closed position when the force from the downstream fluid pressure in the direction of arrow 520 (hereinafter “downstream fluid pressure 520”) exceeds the spring force 518. The position of the spring base 506 within the housing 502 varies between the fully opened position and the fully closed position depending on the balance of spring force 518 and downstream fluid pressure 520. As the downstream fluid pressure 520 applied to the spring base 506 continues to increase, the spring 504 begins to compress when the downstream fluid pressure 520 exceeds the spring force 518. Compression of the spring 504 causes a retraction of the spring base 506 that allows the downstream chamber 503 to expand, which reduces the downstream fluid pressure 520. Movement of the spring base 506 is translated to the plug 512 by the stem 510 so that the pressure regulator passage 514 can be fully sealed when the downstream fluid pressure 520 reaches the set point of the pressure regulator 500. An exemplary set point of the pressure regulator 500 is between 2-4 PSI. When the pressure regulator passage 514 is fully sealed, fluid flow through the pressure regulator 500 is prevented. When the distal fluid pressure 520 falls below the predetermined maximum safe level, the distal fluid pressure 520 exerted on the spring base 506 decreases, which allows the spring 504 to expand and separate plug 512 from the passage 514. Many other embodiments of a pressure regulator that can achieve the same utility are within the scope of the claims.

The stem 510 has a diameter that is smaller than the passage 514 such that fluid may flow through the passage 514 without being substantially impeded by the stem 510. The plug 512 has a diameter that is larger than the passage 514 such that the plug 512 may fully occlude the passage 514 when the valve 508 is in the fully closed position.

During use of the pump 300, the barrel of the syringe 302 is filled with the desired amount of fluid and the syringe 302 is fluidically coupled to the flow path 304. In one example, the user may manually depress the plunger of the syringe 302, thereby creating hydrostatic pressure in the fluid. To permit fluid to flow through the flow path 304, the user may use their thumb or another digit to operate the control valve 400 by moving it to the open position. In another example, the user can open the control valve 400 then depress the plunger of the syringe 302 to begin the flow of fluid. In the open position, fluid is permitted to flow through the flow path 304 and the pressure applied through the syringe 302 causes the fluid the move through the flow path 304, from the syringe 302, through the control valve 400 in the open position, and to the pressure regulator 500. The pressure regulator 500 allows fluid to continue flowing through the flow path 304 at or below the set maximum pressure.

In accordance with the disclosed principles, the disclosed apparatus provides only a limited amount of pressure such that no injury is caused to the vein. Pressure is limited via use of the pressure regulator 500, which provides limited pressurization at a predetermined maximum amount safe for the harvested vein section, thereby eliminating the human error present with manual pressurization during distension of grafted veins. To do so, the spring constant (k) of the pressure regulator 500 is selected to only permit the flow of fluid up to desired or required pressure, with a stiffer spring allowing a higher amount of pressure, while a less stiff spring allowing a lower pressure of the fluid injected into the harvested vein for irrigation. The viscosity of the fluid used in preparing a vein graft can be considered in determining the desired amount of pressure permitted by the pressure regulator 500.

Also, as described above, one embodiment of the pump 300 as disclosed herein includes an on/off mechanism (e.g., the control valve 400) that allows delivery of fluids into the harvested vein to be stopped and started at any time. This stopping capability provides several advantages over the prior art including but not limiting to blocking any unwanted flow due to unintentional depression of the syringe plunger, preventing the creation of a pressure vacuum within the vessel in the event that the syringe plunger is drawn back, and providing a mechanism to immediately stop fluid flow despite a depression of the syringe plunger.

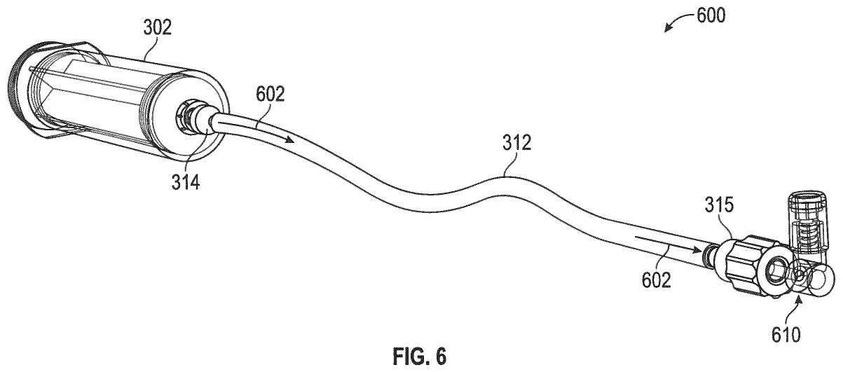

FIG. 6 illustrates another exemplary embodiment of a pressure regulated pump for regulating pressurization of fluid pushed through harvested veins. The pressure regulated pump 600 (hereinafter “pump 600”) controls the pressure of fluid flowing through the vein 312 by a pressure control device 610 located downstream from the vein 312. The pressure control device 610 advantageously provides a safe mechanism for performing vein distension because the user can set the maximum pressure threshold (or threshold value) to a value that will not damage the vein. The maximum pressure threshold (or threshold value) can be between 2-4 PSI. Accordingly, a user introducing the fluid into the vein 312 at a pressure that is too high is no longer fatal to the structural integrity of the vein because pressure control device 610 can dissipate the excess pressure.

The pump 600 depicted in FIG. 6 is formed generally from a syringe 302 coupled to a cannula 314 and a cannula 315 coupled to a pressure control device 700. A vein 312 sutured to the upstream cannula 314 and the downstream cannula 315 completes the flow path 602 that extends from the syringe 302 and through the vein 312 to the pressure control device 610. The pressure control device 610 can be a relief valve that expels fluid from the flow path 602 if the pressure exceeds the pressure set point of the pressure control device 610. The pressure set point of the pressure control device 610 can be selected or set at a limit that prevents damage to the vein 312. An exemplary set point for the pressure control device 610 is 2-4 PSI.

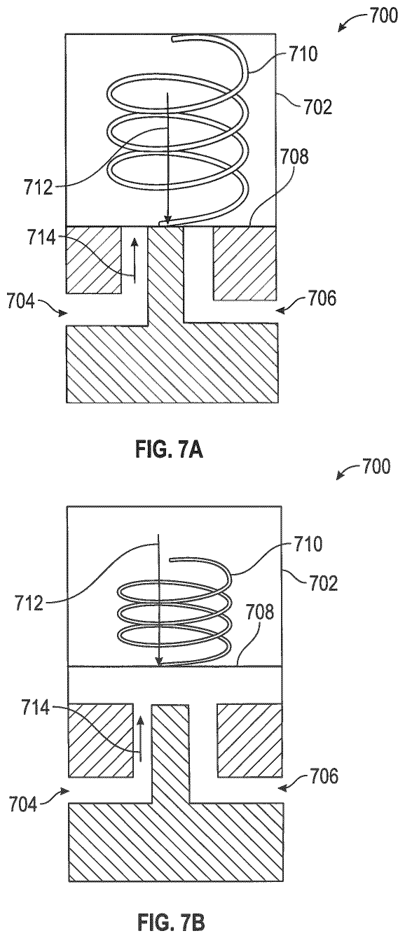

FIGS. 7A and 7B illustrates the principle of operation of pressure control device for use with the pump 600. The illustrative pressure control device is a pressure relief valve 700 and used to control pressure upstream from the pressure relief valve 700 by releasing pressure to the environment in the event that the upstream pressure exceeds a set point of the relief valve 700. The pressure relief valve 700 can be substituted in place of the pressure control device 610 in FIG. 6. Pressure relief valve 700 is meant to be exemplary and non-limiting.

Generally, as seen in FIG. 7A, the pressure relief valve 700 is biased in the closed position by a spring force. Increasing pressure upstream of the pressure relief valve 700 can overcome the spring force causing the pressure relief valve to open, which expels fluid from the pressure relief valve 700 and reduces upstream pressure. To this end, pressure relief valve 700 includes a housing 702 with an inlet 704 separated from an outlet 706 by a spring base 708. The spring base 708 is biased in the closed position by spring 710, which exerts a loading force in the direction illustrated generally by arrow 712 (hereinafter “spring force 712”). As the upstream pressure, represented by arrow 714 (hereinafter “upstream pressure 714”) increases, the spring force 712 is overcome. As seen in FIG. 7B, the spring base 708 assumes the open position, allowing fluid to flow from the opening 704 and through the outlet 706, which reduces the magnitude of the upstream pressure. The spring 710 is configured with a set spring constant (k) which determines the amount of force necessary to compress the spring 710 by a given amount. The spring constant is selected to limit fluid flow through the pressure relief valve 700 to the desired or required maximum proximal fluid pressure. The pressure regulator 700 advantageously provides a safe mechanism for performing vein distension because the user can set the maximum pressure threshold (or threshold value) to a value that will not damage the vein.

With reference to FIGS. 6, 7A, and 7B, during use of the pump 600 outfitted with the pressure relief valve 700 in place of the pressure control device 610, the barrel of the syringe 302 is filled with the desired amount of fluid and the syringe 302 is fluidically coupled to the upstream cannula 314 to connect the syringe 302 to the flow path 602. The user may manually depress the plunger of the syringe 302, thereby creating hydrostatic pressure in the fluid. The pressure applied through the syringe 302 causes the fluid to move through the flow path 602, from the syringe 302, through the first cannula 314, the vein 312, the second cannula 315, and to the pressure relief valve 700. The pressure relief valve 700 allows fluid to be maintained in the vein 312 at or below the set maximum pressure and is configured to release pressure, e.g., expel a portion of the pressurized fluid from the fluid path way 602, in the event that the pressure exceeds the set point of the pressure control device 700.

In some embodiments, the pump 600 includes an optional control valve 400 that controls the flow of fluid through the flow path 602. The control valve 400 is configured to allow the user of pump 300 to manually stop and start the flow of fluid through flow path 602. The control valve 400 can be used to prevent inadvertent discharge of fluid through the vein 312. A particular example of control valve 400 is shown in FIG. 4. Another exemplary control valve is control valve 108 that is shown and described in U.S. Application Ser. No. 63/522,577, the disclosure of which is incorporated herein in its entirety. Other examples of control valve 400 can include but are not limited to ball valves, gate valves, butterfly valves, and globe valves.

The illustrative embodiment depicted in FIG. 6 depicts the fluid source as a syringe 302, i.e., a vessel with a mechanically actuated plunger that can be used to control fluid flow through the fluid flow path 602. However, in another embodiment the syringe 302 can be replaced by a vessel configured to supply pressurized fluid, which obviates the need for the user to depress the plunger of the syringe 302. The control valve 400 can be used as a means to control the flow of fluid through the pump 300.

While this disclosure has been particularly shown and described with reference to preferred embodiments, it will be understood by those skilled in the pertinent field art that various changes in form and detail may be made therein without departing from the spirit and scope of the invention. The inventors expect skilled artisans to employ such variations as appropriate, and the inventors intend the invention to be practiced otherwise than as specifically described herein. Accordingly, this disclosure includes all modifications and equivalents of the subject matter recited in the claims appended hereto, as permitted by applicable law. Moreover, any combination of the above-described elements in all possible variations thereof is encompassed by the disclosure unless otherwise indicated herein or otherwise clearly contradicted by context.

Also, while various embodiments in accordance with the principles disclosed herein have been described above, it should be understood that they have been presented by way of example only, and not limitation. Thus, the breadth and scope of this disclosure should not be limited by any of the above-described exemplary embodiments, but should be defined only in accordance with any claims and their equivalents issuing from this disclosure. Furthermore, the above advantages and features are provided in described embodiments, but shall not limit the application of such issued claims to processes and structures accomplishing any or all of the above advantages.

Additionally, the section headings herein are provided for consistency with the suggestions under 37 C.F.R. 1.77 or otherwise to provide organizational cues. These headings shall not limit or characterize the invention(s) set out in any claims that may issue from this disclosure. Specifically, and by way of example, although the headings refer to a “Technical Field,” the claims should not be limited by the language chosen under this heading to describe the so-called field. Further, a description of a technology as background information is not to be construed as an admission that certain technology is prior art to any embodiment(s) in this disclosure. Neither is the “Summary” to be considered as a characterization of the embodiment(s) set forth in issued claims. Furthermore, any reference in this disclosure to “invention” in the singular should not be used to argue that there is only a single point of novelty in this disclosure. Multiple embodiments may be set forth according to the limitations of the multiple claims issuing from this disclosure, and such claims accordingly define the embodiment(s), and their equivalents, that are protected thereby. In all instances, the scope of such claims shall be considered on their own merits in light of this disclosure, but should not be constrained by the headings set forth herein.

Moreover, the Abstract is provided to comply with 37 C.F.R. § 1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the claims reflect, inventive subject matter lies in less than all features of a single disclosed embodiment. Thus, the following claims are hereby incorporated into the Detailed Description, with each claim standing on its own as a separate embodiment.

Any and all publications, patents, and patent applications cited in this disclosure are herein incorporated by reference as if each were specifically and individually indicated to be incorporated by reference and set forth in its entirety herein.

What is claimed is:

1. A pressure-regulated vein graft preparation pump comprising:
a source of pressurized fluid, wherein the source is configured to supply the pressurized fluid through a flow path extending through a fluid conduit;
a cannula configured to be coupled to an end of the fluid conduit; and
a pressure control device coupled to the cannula, wherein the pressure control device is located downstream from the source of the pressurized fluid and configured to limit a pressure exerted on the fluid conduit by the pressurized fluid.

2. The pressure-regulated vein graft preparation pump of claim 1, wherein the source of the pressurized fluid is a syringe.

3. The pressure-regulated vein graft preparation pump of claim 2, wherein:
the pressure control device is a pressure regulator located upstream of the fluid conduit and configured to terminate the flow of the pressurized fluid when the pressure of the pressurized fluid in the flow path exceeds a threshold value,
the syringe is connected to the pressure regulator, and
the pressure-regulated vein graft preparation pump further comprises a removable clamp sealing a downstream end of the fluid conduit.

4. The pressure-regulated vein graft preparation pump of claim 3, wherein the threshold value is between about 2 pounds per square inch (PSI) to about 4 PSI.

5. The pressure-regulated vein graft preparation pump of claim 3, wherein:
the pressure regulator comprises a housing and a spring-loaded valve disposed within the housing,
the housing comprises a first chamber and a second chamber, and
the spring-loaded valve is configured to form a seal between the first chamber and the second chamber when the pressurized fluid flowing through the flow path fluid exceeds the threshold value.

6. The pressure-regulated vein graft preparation pump of claim 1, further comprising:
a flow control valve disposed in the flow path between the source of the pressurized fluid and the fluid conduit, wherein the flow control valve is configured to regulate a flow of the pressurized fluid through the flow path.

7. The pressure-regulated vein graft preparation pump of claim 6, wherein:
the flow control valve comprises a spring and a plug,
the plug comprises an aperture formed through the plug, and
the plug is biased by the spring to achieve one of a closed position with the aperture misaligned with the flow path or an open position with the aperture aligned with the flow path.

8. The pressure-regulated vein graft preparation pump of claim 7, wherein the flow control valve comprises a guide rail, and wherein the guide rail maintains an orientation of the plug relative to the flow path.

9. The pressure-regulated vein graft preparation pump of claim 2, wherein:
the pressure control device is a pressure relief valve located downstream of the fluid conduit and configured to expel a portion of the pressurized fluid when the pressure of the pressurized in the flow path exceeds a threshold value, and
the syringe is coupled to a cannula coupled to an upstream end of the fluid conduit.

10. The pressure-regulated vein graft preparation pump of claim 9, wherein the threshold value is between about 2 PSI to about 4 PSI.

11. The pressure-regulated vein graft preparation pump of claim 9, wherein:
the pressure relief valve comprises a housing and a spring-loaded valve disposed within the housing,
the housing comprises an inlet and an outlet sealed by the spring-loaded valve, and
the spring-loaded valve is configured to unseal and permit the pressurized fluid to flow from the inlet and through the outlet when the pressure of the pressurized fluid exceeds the threshold value.

12. A method of operating an elastomeric vein graft preparation pump, the method comprising:
coupling a source of pressurized fluid to a flow path extending through a fluid conduit;
coupling a cannula to an end of the fluid conduit;
coupling a pressure control device to the cannula, wherein the pressure control device is located downstream from the source of the pressurized fluid and configured to limit a pressure exerted on the fluid conduit by the pressurized fluid; and
introducing the pressurized fluid into the fluid conduit via the flow path.

13. The method of claim 12, wherein:
the source of the pressurized fluid is a syringe and coupling the source of the pressurized fluid to the flow path further comprises at least partially filling the syringe with a fluid; and
introducing the pressurized fluid into the fluid conduit further comprises depressing a plunger of the syringe to form the pressurized fluid.

14. The method of claim 13, wherein:
the pressure control device is a pressure regulator located upstream from the fluid conduit and configured to terminate the flow of the pressurized fluid when the pressure of the pressurized fluid in the flow path exceeds a threshold value,
coupling the source of the pressurized fluid to the flow path includes coupling the syringe to the pressure regulator, and
the method further comprises sealing a downstream end of the fluid conduit.

15. The method of claim 14, further comprising:
responsive to the pressure exceeding the threshold, terminating the flow of the pressurized fluid through the flow path by the pressure regulator.

16. The method of claim 12, further comprising:
coupling a flow control valve to the flow path between the source of the pressurized fluid and the fluid conduit.

17. The method of claim 16, wherein introducing the pressurized fluid into the fluid conduit further comprises manipulating the flow control valve to permit flow of the fluid through the flow path.

18. The method of claim 13, wherein
the pressure control device is a pressure relief valve located downstream from the fluid conduit and configured expel a portion of the pressurized fluid when the pressure of the pressurized fluid in the flow path exceeds a threshold value,
coupling the syringe to the flow path includes coupling the syringe to a second cannula, and
coupling the second cannula to the fluid conduit.

19. The method of claim 18, further comprising:
responsive to the pressure exceeding the threshold, expelling a portion of the pressurized fluid flowing through the flow path by the pressure relief valve.

20. The method of claim 18, wherein the threshold value is between about 2 PSI to about 4 PSI.
